(19) United States
(12) Patent Application Publication
(10) Pub. No.: US 2026/0326037 A1
(43) Pub. Date: Oct. 22, 2026

(54) FLUID MANAGEMENT TECHNIQUES TO MAINTAIN UNI-DIRECTIONAL PRESSURE GRADIENTS IN PERFUSION SYSTEMS

(71) Applicant: Equilibar, LLC, Fletcher, NC (US)

(72) Inventor: Ryan Matthew Heffner, Asheville, NC (US)

(21) Appl. No.: 19/650,916

(22) Filed: Apr. 17, 2026

Related U.S. Application Data
(60) Provisional application No. 63/790,166, filed on Apr. 17, 2025.

(51) Int. Cl. B01D 63/02

(52) U.S. Cl. CPC B01D 63/02; B01D 2313/18; B01D 2313/243; B01D 2313/903; B01D 2315/10

(57) ABSTRACT

A perfusion filtration apparatus, includes: a bioreactor configured to contain media; a filter apparatus in fluid communication with the bioreactor, the hollow fiber filter apparatus having a retentate side and a permeate side separated by a filter medium; a pump configured to drive flow through the retentate side of the hollow fiber filter apparatus; a first back pressure regulator disposed in fluid communication with the retentate side of the filter apparatus and a first end thereof; a second back pressure regulator disposed in fluid communication with the filter apparatus at either the retentate side at a second end thereof, or the permeate side; and a control system configured to dynamically adjust pressure setpoints of the first and second back pressure regulators to maintain a unidirectional transmembrane pressure gradient across the filter medium throughout each stroke of the pump, thereby preventing reverse transmembrane pressure.

### FIELD OF THE INVENTION

The present invention relates to pressure regulation, and more particularly to pressure regulation in a perfusion filter.

### BACKGROUND OF THE INVENTION

Perfusion filtration has been a common strategy for the pharmaceutical market in upstream manufacturing for the past two decades. This technique has been proven to effectively filter bioreactor media, often called "broth", to act as a fluid exchange and remove used media that cells have polluted and consumed for fresh supplied media. This filtration process is done with a variety of methods involving a filter. This invention will focus on hollow fiber filter designs, which involve crossflow or tangentially flowing media across a retentate port (lumen) through a mode of flow generation. The nature of hollow fiber filters is that there is a laminar flow moving across the filter, with an associated pressure drop following the Hagen Poiseuille pressure drop function.

This pressure drop is typically minimal, with some uses having a 0.5 – 2 psi pressure drop. This will change as the bioreactor process evolves, typically over a 10–30-day period, and fluid density and viscosity change over time.

FIG. 1 illustrates a representative prior art perfusion apparatus 10 having the following basic system components. A bioreactor 12 supplies media "B" which is to be filtered. This is coupled to a filter apparatus 14 by a connecting pipe 16. The filter apparatus 14 extends between first and second ends 18, 20 and includes a filter medium 22 enclosed in a housing 24.

Typically, filter medium 22 is selected to have a pore size smaller than the cells suspended in the broth, to ensure that the filter media filters only cell sizes desired to be retained while allowing the desired smaller sized to pass from retentate to permeate sides. The filter medium 22 may have a closed cross-sectional shape such as annular with its ends closed off by end plates. As a result, the filter medium 22 and housing 24 connectively define an unfiltered space (typically inboard or radially inner) where the broth flows, also referred to as a "retentate" side, and a filtered space (typically outboard or radially outer), also referred to as a "permeate" side.

The second end of the filter apparatus 14 is coupled in fluid communication with an alternating tangential flow (ATF0 pump 26. The ATF pump 26 communicates with the unfiltered space.

The apparatus further includes a collection system on the filtered media side (permeate) to take filtered fluid away from the filter, such as the illustrated permeate pump 25.

An industry common method for generating flow force is to use a diaphragm pump connected to the bottom of the filter. This technique creates a useful benefit, since the stroke of the diaphragm can alternate the flow direction across the filter, which will become useful for de-fouling the membrane.

Since the filter parameters are fixed (excluding restrictions that form as the filter fouls), the resulting system flow and pressure profile will be governed mainly by the pumping control and action. Since bioreactors are not typically pressurized, typical ATF systems (alternating tangential flow) have a feed flow pressure that is low, on the order of single digit psi values or less. This, combined with the permeate port equally being of a low pressure magnitude, can create situations where the pressure gradient in the filter, between the permeate and the retentate, oscillates from positive to negative pressure. This is a known phenomenon which creates a recirculation flow within the filter from retentate to permeate and back, called Starling flow.

Referring to FIG. 2, the dashed line shows the characteristic gauge pressure in the filter apparatus 14, over the length of the filter. It can be seen that the transmembrane pressure differential (TMP) is positive near the first end of the filter, where flow is being introduced. However, the TMP drops over the length of the filter and commonly reverses at the far end of the filter. This reversal in pressure gradient causes the Starling flow described above.

This would mean that the area labeled "X" would have a negative gradient from retentate to permeate (TMP). The negative gradient section means that, relative to the desired direction of flow, the flow would reverse in that section (since flow moves from high to low pressure).

This effect is useful for de-gelling the filter, but ultimately creates non-productive flow, and limits the amount of fluid that can be filtered for a given stroke, therefore reducing the filtration effect on the bioreactor. This creates a need for more filters to be used, or an overall less effective process.

Some studies report that this, from a productive flow perspective, means that permeate flow accounts for only 1-5% of the total flow generated by the flow generating device.

Finally, perfusion being a multi-day to month long process means that there is a long time period as these ATF cycles execute that result in filter fouling. If a solution can be devised to prevent the recirculation of flow unnecessarily, more productive flow for the intention of filtration can result, which may reduce filter area needed (or multiple filters in parallel), filter more effectively, and other similar benefits that would improve future drug processing.

### BRIEF SUMMARY OF THE INVENTION

Described herein are apparatus and methods for maintaining a unidirectional pressure gradient across a tangential flow filter by using synchronized back pressure regulation on the retentate and/or permeate sides of the system during each pump stroke, preventing the formation of reverse transmembrane pressure TMP (Starling flow) and enabling more productive filtration per cycle.

According to an aspect of the present disclosure, a perfusion filtration apparatus includes: a bioreactor configured to contain media; a filter apparatus in fluid communication with the bioreactor, the hollow fiber filter apparatus having a retentate side and a permeate side separated by a filter medium; a pump configured to drive alternating flow through the retentate side of the hollow fiber filter apparatus; a first back pressure regulator disposed in fluid communication with the retentate side of the filter apparatus and a first end thereof; a second back pressure regulator disposed in fluid communication with the filter apparatus at either the retentate side at a second end thereof, or the permeate side; and a control system configured to dynamically adjust pressure setpoints of the first and second back pressure regulators to maintain a unidirectional transmembrane pressure gradient across the filter medium throughout each stroke of the pump, thereby preventing reverse transmembrane pressure.

According to other aspects of the present disclosure, a method of operating a perfusion filtration system includes: providing a filter apparatus in fluid communication with a bioreactor, the filter apparatus having a retentate side and a permeate side separated by a filter medium; driving alternating flow through the retentate side of the filter apparatus using a pump; regulating pressure on the retentate side of the filter apparatus, and a first end thereof, using a first back pressure regulator; regulating pressure either on the retentate side of the filter apparatus at a second end thereof, or the permeate side of the filter apparatus, using a second back pressure regulator; and dynamically adjusting pressure setpoints of the first and second back pressure regulators during each stroke of the pump to maintain a unidirectional transmembrane pressure gradient across the filter medium, thereby preventing reverse transmembrane pressure.

### BRIEF DESCRIPTION OF THE DRAWINGS

The invention may be best understood by reference to the following description taken in conjunction with the accompanying drawing figures in which:

FIG. 1 is a schematic cross-sectional view of a prior art bioreactor and ATF system;

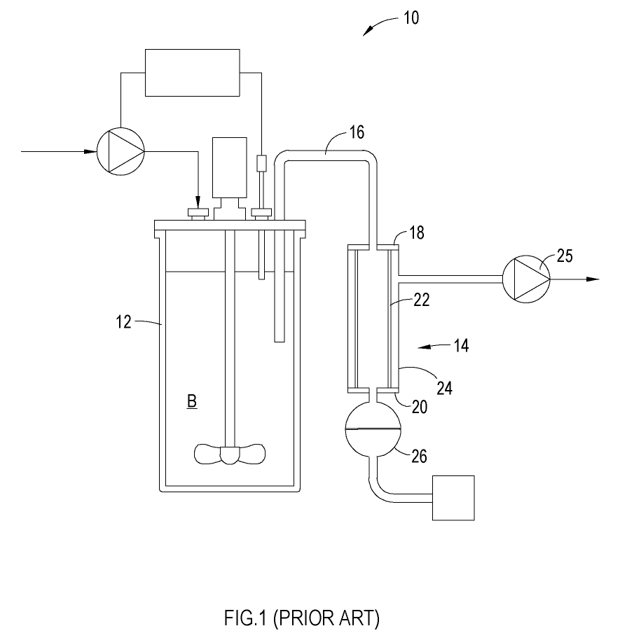

FIG. 2 is a graph illustrating comparative performance of an ATF system described herein to a prior art system;

FIG. 3 is a diagram of a bioreactor with an ATF system incorporating back pressure regulators to control pressure gradient;

FIG. 4. is a schematic cross-sectional view of a dome-loaded direct sealing back pressure regulator;

FIG. 5 is a diagram of a bioreactor with an rTFF system incorporating back pressure regulators to control pressure gradient;

FIG. 6 is a diagram of a bioreactor with a single-pass tangential flow filtration system incorporating back pressure regulators to control pressure gradient; and

FIG. 7 is a diagram of a dome-loaded back pressure regulator coupled to a three-port valve and a pair of pilot pressure regulators.

### DETAILED DESCRIPTION OF THE INVENTION

The present invention relates to methods and apparatus for maintaining a unidirectional pressure gradient across a tangential flow filter by using synchronized back pressure regulation on the retentate and/or permeate sides of the system during each pump stroke, preventing the formation of reverse transmembrane pressure TMP (Starling flow) and enabling more productive filtration per cycle.

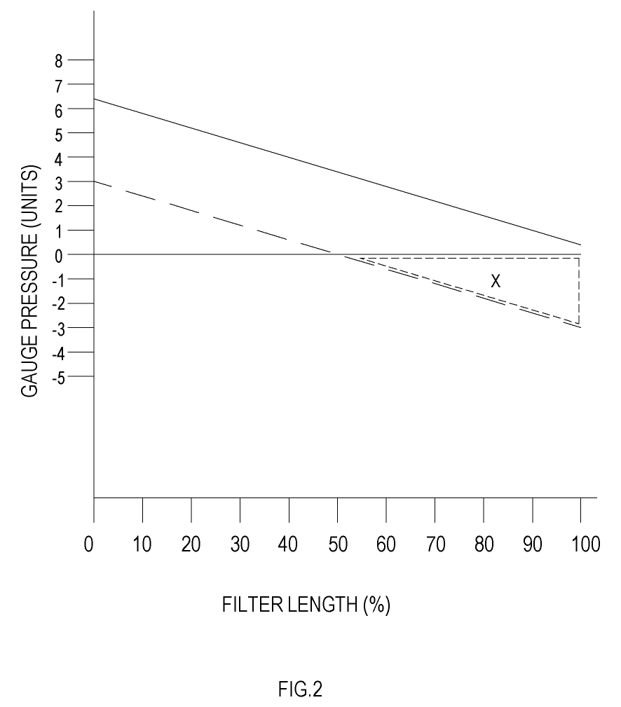

A pressure regulator, such as a diaphragm pressure regulator, can be implemented as described in the examples below to prevent Starling flow, which is a large source of wasted energy / non-productive flow. The control setpoints, along with the timing strategy, would be unique to each end use and the product needs. However, all embodiments may adopt a main principle of oscillating a setpoint of a pressure regulator from a positive to zero or negative gauge pressure to manipulate the pressure gradient across the filter, relative to 0 psig, to always be in a positive or negative direction. This result would be represented by the solid line in the graph of FIG. 2. as a representative depiction only. This would mean that, in contrast to the prior art method, the area labeled X would not have a negative gradient from retentate to permeate (TMP).

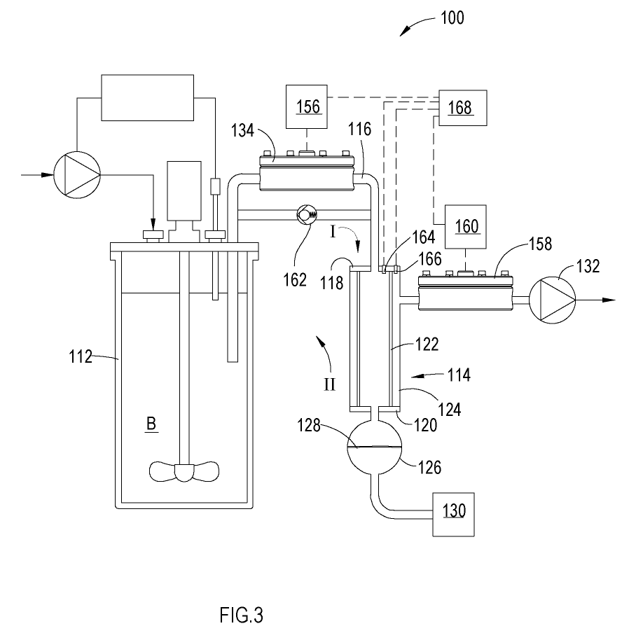

FIG. 3 illustrates apparatus 100 similar in construction to apparatus 10 described above. It includes bioreactor 112 which supplies media (e.g. "broth") B which is to be filtered. This is coupled to a filter apparatus 114 by a connecting pipe 116. The filter apparatus 114 extends between first and second ends 118, 120 and includes a filter medium 122 enclosed in a housing 124.

The filter medium 122 may have a closed cross-sectional shape such as an annulus with its ends closed off by end plates. As a result, the filter medium 122 and housing 124 connectively define an unfiltered space (typically inboard or radially inner) where the broth flows, alternatively referred to as a "retentate side", and a filtered space (typically outboard or radially outer), alternative referred to as a "permeate side". The filter medium 122 may comprise a hollow fiber medium.

The second end 120 of the filter apparatus 114 is coupled in fluid communication with an ATF pump 126. The ATF pump 126 communicates with the unfiltered space (retentate side). The ATF pump 126 has a pump chamber including an internal diaphragm 128. One side of the diaphragm 128 is connected in fluid communication with a controller 130. This is operable to apply fluid pressure or vacuum as desired, to either draw the diaphragm 128 towards the controller 130, causing fluid to flow from the reactor 112 through the filter apparatus 114 and into the pump chamber, (referred to as a "forward flow direction", see arrow "I") or alternately to distend the diaphragm 128 towards the filter apparatus 114, causing the fluid to flow from the ATF pump 128 through the filter apparatus 114 back towards the reactor 112 (referred to as a "reverse flow direction", see arrow "II").

The apparatus 100 includes a collection system on the filtered media side (permeate) to take filtered fluid away from the filter apparatus 114, such as the illustrated permeate pump 132. The permeate pump 132 is connected in fluid communication with the filtered space of the filter apparatus 114.

A first pressure regulator 134 is disposed inline in the connecting pipe 116. In a preferred embodiment, the first pressure regulator 134 may be a dome-loaded direct sealing pressure regulator. As explained in more detail below, in the forward flow direction, no action from the first pressure regulator 134 is needed, but in the reverse flow direction, the first pressure regulator 134 functions to increase pressure in the unfiltered space of the filter apparatus 114.

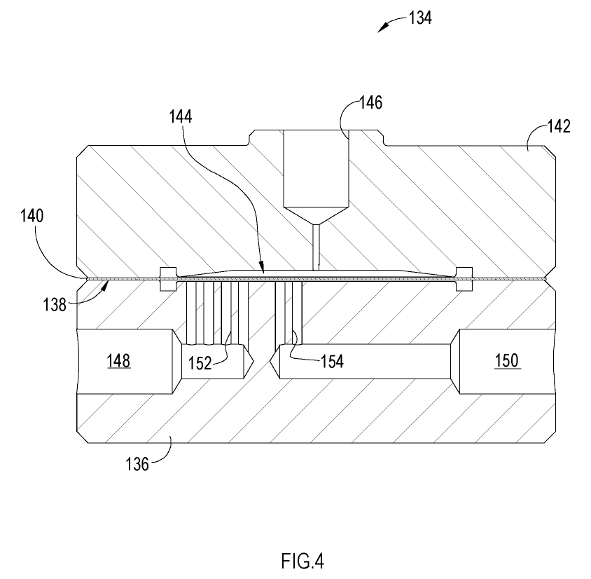

A simplified diagram of this type of back pressure regulator is shown in FIG. 4. Back pressure regulator 134 (alternatively referred to as a "BPR" herein) includes a body 136 including a process surface 138. A diaphragm 140 (also referred to interchangeably herein as a membrane) made of flexible material such as PTFE or fiber reinforced PTFE sheeting is disposed adjacent to the process surface 138. The diaphragm 140 has opposed sides referred to as reference and process sides, with the process side facing the process surface 138. The perimeter of the diaphragm 140 is secured against the body 136 by a reference housing 142 which is attached to the body 136. Collectively, a space defined between the diaphragm 140 and the reference housing 142 is referred to as a "dome" 144. A reference port 146 is formed in the reference housing 142 and is disposed in fluid communication with the reference side of the diaphragm 140. Inlet and outlet ports 148, 150, respectively, are also formed in the body 136. At least one inlet orifice 152 is disposed in fluid communication with the inlet port 148 and the process surface 138. At least one outlet orifice 154 is disposed in fluid communication with the outlet port 150 and the process surface 138. The orifices are small holes or small openings. The first pressure regulator 134 is installed with its inlet port 148 towards the filter apparatus 114.

The back pressure regulator 134 is a fast acting device that can natively respond to fluid transients, and can manage vacuum regulation when required.

Direct diaphragm sealing back pressure regulators of this type are commercially available from Equilibar, LLC (Fletcher, NC). Such direct diaphragm sealing BPRs use a "1:1" pilot pressure (typically compressed air, also optionally vacuum) on the dome, and use pneumatic electronic pressure regulators (EPR, or I/P’s) to provide the pilot pressure. An EPR 156 is shown schematically in FIG. 3. In such systems, the EPR contains a high speed proportional-integral-derivative (PID) control (or other similar control) loop which constantly adjusts its output air pressure to match that of an input signal.

The construction and materials of the back pressure regulator 134 (as well as the other components of apparatus 100) may be "multi-use" (i.e., reusable) or "single-use" (i.e., disposable).

With continued reference to FIG. 3, a second pressure regulator 158 is disposed inline between the filter housing 124 and the permeate pump 132. This may be a dome-loaded direct sealing BPR as described above. An electronic pressure regulator 160 is coupled to the second pressure regulator 158. The second pressure regulator 158 is installed with its inlet port towards the filter apparatus 114.

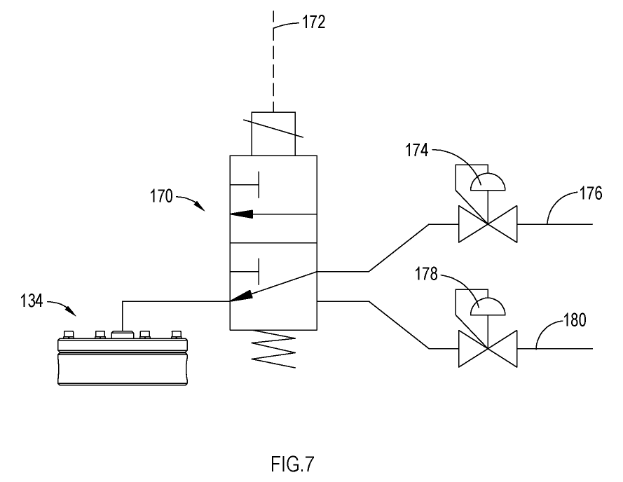

An alternative mechanism that may be substituted for any of the EPRs for providing simple alternation of pressures is shown in FIG. 7. The reference port of the back pressure regulator 104 is coupled to an outlet port of a three-port divert valve 170 (shown schematically). As illustrated, this may be solenoid-operated and may be connected to a control line 172. A first manually-set pressure regulator 174 is coupled to a first inlet port of the valve 170 and to a first fluid reference 176 (e.g., compressed air or vacuum). A second manually-set pressure regulator 178 is coupled to a second inlet port of the valve 170 and to a second fluid reference 180 (e.g.., compressed air or vacuum). If both reference pressures will be pressure or vacuum, a common fluid reference may be used for both regulators 174, 178. Said pressure regulator system would have means to accommodate a quick fill and relief of air from the domes of the regulators by using either a small volume (capacitance), bleed, or a pressure relief capability

In the illustrated system, the prevailing pressure differences in the forward flow mode may cause the first pressure regulator 134 to block flow. Accordingly, a check valve 162 may be provided in parallel with the first pressure regulator to permit flow in the direction of arrow I. Alternatively, the check valve 162 may be omitted, and during periods of forward flow, the first pressure regulator 134 would be supplied with a low positive pressure setpoint (or vacuum) by the EPR 156 to force it to open.

In the forward flow mode, pressure at the permeate side of the filter media 122 would be modulated by the second pressure regulator 158.

In the illustrated system, in a reverse flow mode, the ATF pump 126 would be reversed in order to motivate flow in the direction of arrow II. In this flow mode, the first pressure regulator 134 would be used to regulate pressure in the unfiltered space and the second pressure regulator 158 would be used to regulate pressure in the filtered space (permeate side).

In FIG. 3, the pressure regulators 134, 158 would oscillate in pilot pressure. The pilot pressures could be set to a negative gauge pressure. This would be useful, since the permeate pump 132 can pull a strong vacuum if set to the appropriate RPM, which would allow the regulator 158 when given a vacuum pressure to adjust the permeate pressure to a vacuum level that would still allow for consistent uni-directional pressure gradients in an ATF setup.

Optionally, apparatus 100 may further include a control system including: one or more pressure sensors 164 on the retentate permeate side of the filter media 122, one or more pressure sensors 166 on the permeate side of the filter media 122, and an electronic controller 168 configured to adjust pilot pressures to the pressure regulators 156, 160 in response to pressure signals from the sensors 164, 166. The controller 168 may be configured or programmed to adjusts the pilot pressures to prevent any region of the filter apparatus 114 from experiencing a negative transmembrane pressure.

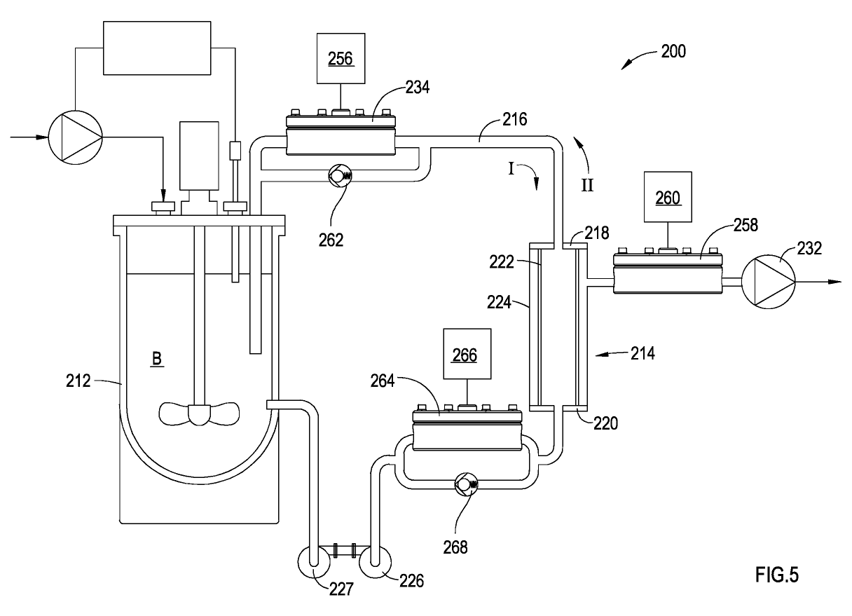

FIG. 5 illustrates apparatus 200 similar in construction to apparatus 100 described above, a distinction being that the apparatus 200 is implemented on a reverse TFF (rTFF) apparatus. It includes bioreactor 212 which supplies media (e.g. "broth") B which is to be filtered. This is coupled to a filter apparatus 214 by a connecting pipe 216. The filter apparatus 214 extends between first and second ends 218, 220 and includes a filter medium 222 enclosed in a housing 224.

The filter medium 222 may have a closed cross-sectional shape such as annular with its ends closed off by end plates. As a result, the filter medium 222 and housing 224 connectively define an unfiltered space (typically inboard or radially inner) alternatively referred to as a "retentate side", and a filtered space (typically outboard or radially outer), alternative referred to as a "permeate side". The filter medium 222 may comprise a hollow fiber medium.

The second end 220 of the filter apparatus 214 is coupled in fluid communication with a first rTFF pump 226 and a second rTFF pump 227. The rTFF pumps 226, 227 communicate with the unfiltered space. The first rTFF pump 226, for example a centrifugal pump, is configured to pump fluid in a first direction depicted by arrow I ("forward flow direction"). The second rTFF pump 227, for example a centrifugal pump, is configured to pump fluid in a second direction depicted by arrow II ("reverse flow direction").

The apparatus 200 includes a collection system on the filtered media side (permeate) to take filtered fluid away from the filter apparatus 214, such as the illustrated permeate pump 232. The permeate pump 232 is connected in fluid communication with the filtered space of the filter apparatus 214.

A first pressure regulator 234 is disposed inline in the connecting pipe 216. In a preferred embodiment, the first pressure regulator 234 may be a dome-loaded direct sealing pressure regulator. An electronic pressure regulator 256 is coupled to the first pressure regulator 234. The first pressure regulator 234 is installed with its inlet port towards the filter apparatus 214.

A second pressure regulator 258 is disposed inline between the filter housing 224 and the permeate pump 232. This may be a dome-loaded direct sealing BPR as described above. An electronic pressure regulator 260 is coupled to the second pressure regulator 258. The second pressure regulator 258 is installed with its inlet port towards the filter apparatus 214.

A third pressure regulator 264 is disposed inline between the filter housing 224 and the rTFF pumps 226, 227. This may be a dome-loaded direct sealing BPR as described above. An electronic pressure regulator 266 is coupled to the third pressure regulator 264. The third pressure regulator 264 is installed with its inlet port towards the filter apparatus 214.

In the illustrated system, the prevailing pressures in the forward flow mode may cause the first pressure regulator 234 to block flow. Accordingly, a first check valve 262 may be provided in parallel with the first pressure regulator 234 to permit flow in the direction of arrow "I". Alternatively, the first check valve 262 may be omitted, and during periods of forward flow, the first pressure regulator 234 would be supplied with a low positive pressure setpoint (or vacuum) by the EPR 256 to force it to open.

In the forward flow mode, pressure at the downstream end of the unfiltered side of the filter media 222 (i.e., second end 220) would be modulated by the third pressure regulator 264. In the forward flow mode, pressure at the permeate side of the filter media 222 would be modulated by the second pressure regulator 258.

In the illustrated system, in a reverse flow mode, the first rTFF pump 226 would be shut down and the second rTFF pump 227 operated in order to motivate flow in the direction of arrow "II". In this flow mode, the first pressure regulator 234 would be used to regulate pressure at the downstream end of the unfiltered side of the filter media 222 (i.e., first end 218). In the reverse flow mode, pressure at the permeate side of the filter media 222 would be modulated by the second pressure regulator 258.

In the reverse flow mode, prevailing pressures may cause the third pressure regulator 264 to block flow. Accordingly, a second check valve 268 may be provided in parallel with the third pressure regulator 264to permit flow in the direction of arrow "II". Alternatively, the second check valve 268 may be omitted, and during periods of forward flow, the third pressure regulator 264 would be supplied with a low positive pressure setpoint (or vacuum) by the EPR 266 to force it to open.

In FIG. 5, the pressure regulators 234, 258, 264 would oscillate in pilot pressure, but may to be operated in a 0 – X psia format, meaning the pilot pressures could be set to a negative gauge pressure. This would be useful, since the permeate pump 232 can pull a strong vacuum if set to the appropriate RPM, which would allow the regulator 258 when given a vacuum pressure to adjust the permeate pressure to a vacuum level that would still allow for consistent uni-directional pressure gradients in an ATF setup.

Optionally, apparatus 200 may further include a control system (not illustrated) with retentate and permeate pressure sensors and a controller as described above for apparatus 100.

The concepts described herein are also applicable to "single-pass" filtration systems which do not have alternating or reversing flow. Examples include single-pass tangential flow filtration systems.

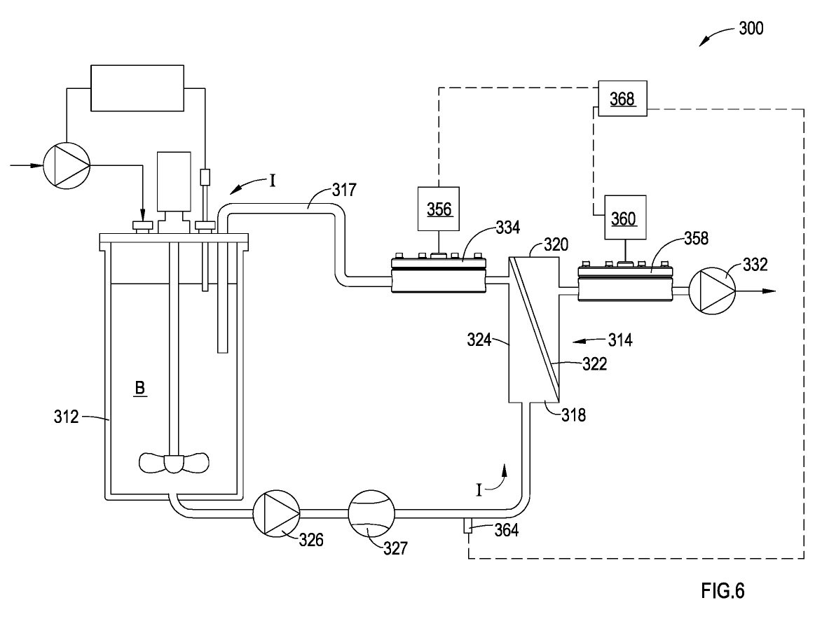

FIG. 6 illustrates apparatus 300 similar in construction to apparatus 10 described above. It includes bioreactor 312 which supplies media (e.g. "broth") B which is to be filtered. This is coupled to a filter apparatus 314 by a supply pipe 316. The filter apparatus 314 extends between first and second ends 318, 320 and includes a filter medium 322 enclosed in a housing 324. A return pipe 317 interconnects the filter apparatus 314 with the reactor 312.

The filter medium 322 divides the interior of the housing 324 into an unfiltered space where the broth flows, alternatively referred to as a "retentate side", and a filtered space, alternatively referred to as a "permeate side".

The first end 318 of the filter apparatus 114 is coupled in fluid communication with a circulating pump 326. The circulating pump 326 communicates with the unfiltered space (retentate side). The circulating pump 126 motivates flow in the direction "I", causing the fluid to flow from the reactor 312 through the circulating pump 326, through the filter apparatus 314 before returning to the reactor 312. The apparatus 300 may incorporate a flowmeter 327 inline with the pump 326.

The apparatus 300 includes a collection system on the filtered media side (permeate) to take filtered fluid away from the filter apparatus 314, such as the illustrated permeate pump 332. The permeate pump 332 is connected in fluid communication with the filtered space of the filter apparatus 314.

A first pressure regulator 334 is disposed inline in the return pipe 317. In a preferred embodiment, the first pressure regulator 134 may be a dome-loaded direct sealing pressure regulator as described above. An electronic pressure regulator 356 is coupled to the first pressure regulator 334. The first pressure regulator 3344 is installed with its inlet port towards the filter apparatus 314.

With continued reference to FIG. 6, a second pressure regulator 358 is disposed inline between the filter housing 324 and the permeate pump 332. This may be a dome-loaded direct sealing BPR as described above. An electronic pressure regulator 360 is coupled to the second pressure regulator 358. The second pressure regulator 358 is installed with its inlet port towards the filter apparatus 314.

In operation, the first pressure regulator 334 would be used to regulate pressure in the unfiltered space and the second pressure regulator 358 would be used to regulate pressure in the filtered space (permeate side).

In FIG. 3, the pressure regulators 134, 158 would oscillate in pilot pressure. The pilot pressures could be set to a negative gauge pressure. This would be useful, since the permeate pump 132 can pull a strong vacuum if set to the appropriate RPM, which would allow the regulator 158 when given a vacuum pressure to adjust the permeate pressure to a vacuum level that would still allow for consistent uni-directional pressure gradients in an ATF setup.

Optionally, apparatus 300 may further include a control system including one or more pressure sensors 364 and an electronic controller 368 configured to adjust pilot pressures to the pressure regulators 356, 360 in response to pressure signals from the sensors 364. The controller 368 may be configured or programmed to adjust the pilot pressures to prevent any region of the filter apparatus 314 from experiencing a negative transmembrane pressure.

Other similar applications that may benefit from the pressure regulation methods described above are: N-1 perfusion and high-viscosity harvest (e.g., mAb, CHO broth).

The apparatus described above is capable of regulating pressure in an absolute or gauge sense when connected to a vacuum source (not depicted in schematics above). Given the low pressure drop for certain filter selections, this embodiment must be accurate at controlling pressure in the 0.0X psi range to optimize on pressure regulation while avoiding unnecessary energy required to generate flow or unproductive flow from Starling flow due to stiction or hysteresis in the regulator valve.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

What is claimed is:

1. A perfusion filtration apparatus, comprising:
a bioreactor configured to contain media;
a filter apparatus in fluid communication with the bioreactor, the hollow fiber filter apparatus having a retentate side and a permeate side separated by a filter medium;
a pump configured to drive flow through the retentate side of the hollow fiber filter apparatus;
a first back pressure regulator disposed in fluid communication with the retentate side of the filter apparatus and a first end thereof;
a second back pressure regulator disposed in fluid communication with the filter apparatus at either the retentate side at a second end thereof, or the permeate side; and
a control system configured to dynamically adjust pressure setpoints of the first and second back pressure regulators to maintain a unidirectional transmembrane pressure gradient across the filter medium throughout each stroke of the pump, thereby preventing reverse transmembrane pressure.

2. The apparatus of claim 1, wherein at least one of the first and second back pressure regulators is a dome-loaded direct sealing back pressure regulator comprising a body having a process surface, a diaphragm disposed adjacent to the process surface, a reference housing secured to the body and defining a dome on a reference side of the diaphragm, and a reference port in fluid communication with the dome.

3. The apparatus of claim 2, further comprising an electronic pressure regulator coupled to the dome of the dome-loaded direct sealing back pressure regulator, the electronic pressure regulator configured to supply a pilot pressure to the dome, wherein the electronic pressure regulator includes a proportional-integral-derivative control loop configured to adjust an output pressure to match an input signal.

4. The apparatus of claim 1, wherein the pump is a diaphragm pump having an internal diaphragm, and a controller operable to apply fluid pressure or vacuum to the internal diaphragm to alternately draw fluid from the bioreactor through the filter apparatus into a pump chamber and to expel fluid from the pump chamber back through the filter apparatus toward the bioreactor.

5. The apparatus of claim 1, further comprising a permeate pump in fluid communication with the permeate side of the filter apparatus, the permeate pump configured to draw filtered media away from the hollow fiber filter apparatus.

6. The apparatus of claim 1, further comprising a check valve disposed in parallel with the first back pressure regulator, the check valve configured to permit flow in a forward flow direction from the bioreactor toward the hollow fiber filter apparatus.

7. The apparatus of claim 1, wherein the control system comprises:
one or more pressure sensors disposed on the retentate side and the permeate side of the filter apparatus; and
a controller configured to adjust pilot pressures supplied to the first and second back pressure regulators in response to pressure signals received from the one or more pressure sensors.

8. The apparatus of claim 7, wherein the controller is configured to adjust the set-point of at least one of the back pressure regulators to minimize the occurrence of negative transmembrane pressure

9. A method of operating a perfusion filtration system, comprising:
providing a filter apparatus in fluid communication with a bioreactor, the filter apparatus having a retentate side and a permeate side separated by a filter medium;
driving flow through the retentate side of the filter apparatus using a pump;
regulating pressure on the retentate side of the filter apparatus, and a first end thereof, using a first back pressure regulator;
regulating pressure either on the retentate side of the filter apparatus at a second end thereof, or the permeate side of the filter apparatus, using a second back pressure regulator; and
dynamically adjusting pressure setpoints of the first and second back pressure regulators during each stroke of the pump to maintain a unidirectional transmembrane pressure gradient across the filter medium, thereby minimizing reverse transmembrane pressure.

10. The method of claim 9, wherein dynamically adjusting the pressure setpoints comprises oscillating a pilot pressure of at least one of the first and second back pressure regulators between a positive gauge pressure and a zero or negative gauge pressure.

11. The method of claim 9, wherein the first and second back pressure regulators are dome-loaded direct sealing back pressure regulators, and wherein dynamically adjusting the pressure setpoints comprises supplying pilot pressures to domes of the first and second back pressure regulators via respective electronic pressure regulators.

12. The method of claim 9, further comprising drawing filtered media from the permeate side of the filter apparatus using a permeate pump.

13. The method of claim 9, wherein driving alternating flow comprises operating a diaphragm pump to alternate a direction of flow through the retentate side of the filter apparatus between a forward flow direction and a reverse flow direction.

14. The method of claim 9, further comprising:
sensing pressures on the retentate side and the permeate side of the hollow fiber filter apparatus using one or more pressure sensors; and
adjusting set-points of at least one of the back pressure regulators to minimize the occurrence of negative transmembrane pressure.

15. The method of claim 9, wherein during a forward flow mode, the first back pressure regulator is supplied with a low positive pressure setpoint or a vacuum setpoint to permit flow in the forward flow direction.
